(19) United States
(12) Patent Application Publication
(10) Pub. No.: US 2024/0226497 A1
(43) Pub. Date: Jul. 11, 2024

(54) SLEEP MANAGEMENT SYSTEM, SLEEP MANAGEMENT METHOD, CONTROL PROGRAM, AND STORAGE MEDIUM

(71) Applicant: KYOCERA Corporation, Kyoto-shi, Kyoto (JP)

(72) Inventor: Shinya NAGASAKI, Kyoto-shi, Kyoto (JP)

(21) Appl. No.: 18/572,120

(22) PCT Filed: Jun. 13, 2022
(86) PCT No.: PCT/JP2022/023568

§ 371 Date: Dec. 19, 2023

(30) Foreign Application Priority Data
Jun. 29, 2021 (JP) 2021-108103

(51) Int. Cl. A61M 21/00

(52) U.S. Cl. CPC A61M 21/00; A61M 2021/0083

(57) ABSTRACT

A sleep management system according to an aspect of the present disclosure includes a first determiner configured to determine awakening timing of a user and an awakener configured to notify the user of the awakening timing in accordance with a determination result of the first determiner. In the sleep management system, a second determiner is able to determine the awakening timing in accordance with a second index when a trouble occurs in determination of the awakening timing based on a first criterion.

### TECHNICAL FIELD

The present disclosure relates to a sleep management system for managing sleep of a user, a sleep management method, a control program, and a storage medium.

### BACKGROUND OF INVENTION

Patent Document 1 discloses a technology for detecting a sleep state.

### CITATION LIST

### Patent Literature

Patent Document 1: JP 2018-161432 A

### SUMMARY

A sleep management system according to an aspect of the present disclosure includes: a determiner configured to determine awakening timing of a user; and an awakener configured to notify the user of the awakening timing in accordance with a determination result of the determiner. The determiner is able to determine the awakening timing in accordance with a second index when a trouble occurs in determination of the awakening timing based on a first index.

A sleep management method according to an aspect of the present disclosure is a sleep management method in a sleep management system including a determiner. The method includes: by the determiner, determining awakening timing of a user in accordance with a first index; and determining the awakening timing of the user in accordance with a second index when a trouble occurs in the determining the awakening timing based on the first index.

Each step of the sleep management method according to each aspect of the present disclosure may be implemented by a computer. In this case, a control program of the sleep management system that causes a computer to implement the sleep management method by causing the computer to operate as each unit (software element) included in the sleep management system and a computer-readable recording medium in which the control program is recorded are also included in the scope of the present disclosure.

### BRIEF DESCRIPTION OF THE DRAWINGS

FIG. 1 is a block diagram illustrating an example of a configuration of a sleep management system according to an embodiment.

FIG. 2 is a schematic view illustrating an appearance of a device in the sleep management system of FIG. 1.

FIG. 3 is a flowchart illustrating an example of a flow of an operation of a NAP function performed in the sleep management system of FIG. 1.

FIG. 4 is a flowchart illustrating another example of the flow of the operation of the NAP function performed in the sleep management system of FIG. 1.

FIG. 5 is a block diagram illustrating an example of a configuration of a sleep management system according to another embodiment.

FIG. 6 is a block diagram illustrating an example of a configuration of a sleep management system according to another embodiment.

### DESCRIPTION OF EMBODIMENTS

Determining a sleep state of a user and improving reliability of sleep management have been awaited. An aspect of the present disclosure can improve reliability of sleep management.

### First Embodiment

### Overview of Sleep Management System 100

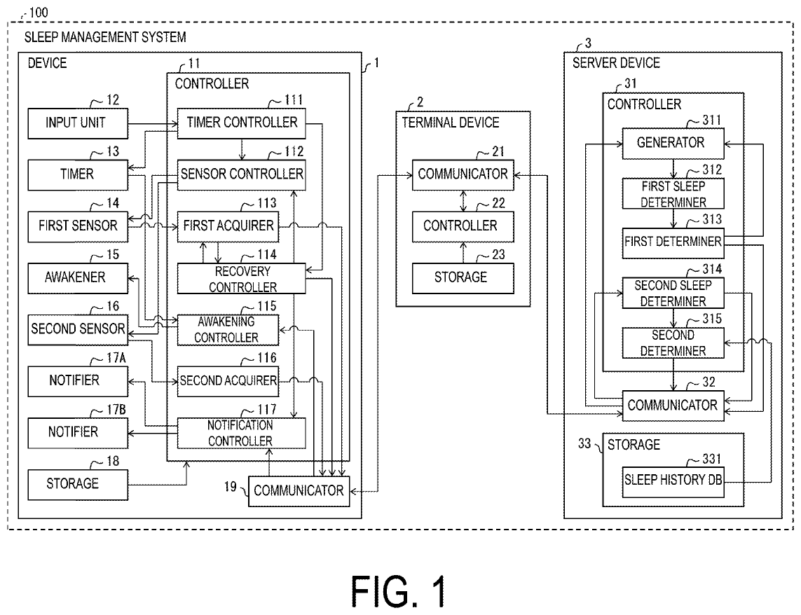

Hereinafter, an overview of a sleep management system 100 according to an embodiment of the present disclosure will be described. FIG. 1 is a block diagram illustrating an example of a configuration of the sleep management system 100 according to an embodiment.

As illustrated in FIG. 1, the sleep management system 100 includes a device 1 (first device), a terminal device 2, and a server device 3 (second device). The device 1, the terminal device 2, and the server device 3 may be separate devices. The sleep management system 100 has a sleep management function to manage sleep (nap) of a user by encouraging the user to fall asleep and awakening the user who has fallen asleep at appropriate timing. The sleep management function executed in the sleep management system 100 may also be referred to as a NAP function. The term “sleep” herein may refer to a short nap or a long sleep usually at night.

An outline of sleep management performed in the sleep management system 100 will be described below. First, when an operation of the NAP function is started by a user's operation, the device 1 acquires first information related to a user's physiological state and transmits the first information to the server device 3. The server device 3 generates a first index on the basis of the first information, and determines awakening timing of the user on the basis of whether a sleep state of the user determined on the basis of the first index satisfies a preset first criterion. The device 1 encourages the user to wake up by notifying the user of the awakening timing when the determined awakening timing arrives.

As described above, the user can wake up at appropriate timing after falling asleep. Therefore, by using the sleep management system 100, the user can sleep with high satisfaction even for a short sleep period.

The first index is an index used to determine whether the user is in a sleep state. For example, the first index may be biological information on the user. For example, the first index may be information related to a blood flow of the user. More specifically, the first index is information indicating an amount of blood flow of the user, waveform data of a pulse wave of the user, or the like. The first information is information used to generate the first index. When the first index is waveform data of a pulse wave of the user, the first information is, for example, a value of a sensor signal reflecting a physiological state of the user. More specifically, the first information may be a sensor signal value corresponding to the intensity of scattered light obtained when light emitted to the user is scattered by blood vessels, blood cells, and the like of the user. When the device 1 acquires the first information multiple times, the amount of blood flow of the user is calculated as the first index.

The first criterion is a criterion used to specify the awakening timing of the user. The sleep management system 100 may determine (estimate) the awakening timing of the user by determining whether the sleep state of the user determined based on the first index satisfies the first criterion. For example, the sleep management system 100 may determine timing at which the sleep state of the user matches the first criterion as the awakening timing of the user. The first criterion may be a criterion based on biological information on the user. For example, the first criterion may be a criterion related to a sleep state of the user. As an example, the first criterion may be indicative of a stage of sleep, specifically stage 2 to 3 of non-REM sleep.

The sleep management system 100 has a recovery function to attempt to resolve a fatal error when the error occurs in the device 1. The fatal error is an error indicating a state in which the device 1 cannot normally acquire or transmit the first information. The fatal error will be described in detail below. When a fatal error occurs in the device 1, the server device 3 cannot receive the first information and cannot determine the sleep stage based on the first index. Therefore, when a fatal error occurs in the device 1, in other words, when a trouble occurs in the determination of the sleep state based on the first index in the sleep management system 100, the server device 3 cannot determine the awakening timing of the user.

When the above-described error occurs, the sleep management system 100 can determine the awakening timing of the user on the basis of a second index different from the first index, and notify the user of the timing. The server device 3 determines whether the user is awake based on a third index, for example, information indicating a degree (acceleration) of motion of the user, and when the user is awake, the server device notifies the user to encourage the user to restart the device 1. This allows the user to recognize that an error has occurred in the device 1. The device 1 is restarted by a user's operation, so that the error can be resolved.

As a result of the above determination, when the user has already fallen asleep, the server device 3 determines the awakening timing of the user on the basis of the second index. The device 1 can notify the user of the determined awakening timing at the awakening timing. The second index is an index used to determine the awakening timing of the user. The second index may be, for example, a criterion based on non-biological information. The second index may be, for example, a criterion related to a sleep period. Based on the second index, the sleep management system 100 may specify time from when the user falls asleep until when the awakening timing is notified, i.e., a sleep period of the user.

As described above, when a fatal error occurs in the device 1, if the user is awake, the sleep management system 100 can attempt recovery from the error by encouraging the user to restart the device 1. When the user has already fallen asleep, the user is encouraged to wake up at the awakening timing determined based on the degree of motion of the user, and thus the user can be encouraged to wake up at appropriate timing even when the determination based on the waveform data of the pulse wave of the user cannot be made.

Hereinafter, a case in which the sleep management system 100 acquires information related to a physiological state of a user as the first information and generates information indicating an amount of blood flow of the user as the first index on the basis of the information will be described as an example. In the following description, a case in which the sleep management system 100 uses, as the second index, an elapsed period after the NAP function is turned on is used as an example. A configuration in which an index related to a motion of the user is used as the third index is used as an example. The index related to the motion of the user is, for example, an index indicating a degree of the motion of the user, and specifically, acceleration or the like based on the motion of the user. However, the first information, the first index, the second index, and the third index are not limited to those described above.

For example, the first index may be data including various variables calculated on the basis of a blood flow. Specifically, for example, the first index may include data related to at least one of an amount of blood flow, a heart rate, a heart beat interval, a cardiac output, a blood flow wave height, and a coefficient of variation of blood vessel motion (vasomotion) of the user.

The amount of blood flow represents the amount of blood flowing in a blood vessel per unit time. The heart rate represents the number of beats of the heart per unit time. The heart beat interval represents an interval between beats of the heart. Cardiac output represents the amount of blood delivered in one beat of the heart. The blood flow wave height represents a difference between the maximum value and the minimum value of the amount of blood flow in one beat of the heart. Vasomotion represents a contraction-expansion movement of the blood vessel that occurs spontaneously and rhythmically. The coefficient of variation of vasomotion represents a value indicating, as a variation, the change in the amount of blood flow occurring on the basis of the vasomotion.

### Device 1

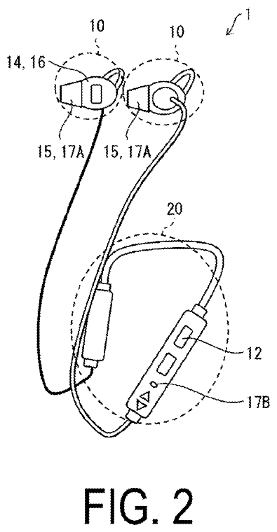

FIG. 2 is a schematic view illustrating an appearance of the device 1. First, a configuration of the device 1 will be described using FIGS. 1 and 2. As described above, the device 1 acquires the first information from the body of the user and transmits the information to the server device 3 via the terminal device 2. The device 1 performs an operation of encouraging the user to wake up and various notifications to the user in response to the information received from the server device 3 via the terminal device 2. The device 1 is communicably connected to the terminal device 2.

The device 1 acquires the first information from the body of the user by being worn on the body of the user. As an example, as illustrated in FIG. 2, the device 1 may be an integrated wireless earbud shaped device that may be worn in a user's ear. The device 1 includes a wearing portion 10 to be worn on the body of the user, and a neck hanging portion 20 that can be positioned around the neck of the user in a state where the wearing portion 10 is worn on the user. However, the shape of the device 1 is not limited to that illustrated in FIG. 2. For example, the device 1 may be a device having a separate type wireless earbud shape. In this case, the device 1 may include only the wearing portion 10.

The wearing portion 10 is a member to be worn on the body of the user. As an example, the wearing portion 10 can be inserted into the ear of the user, in particular into the ear hole. The wearing portion 10 includes a first sensor 14, an awakener 15, a second sensor 16, and a notifier 17A. As illustrated in FIG. 2, the device 1 may include the two wearing portions 10. However, the number of the wearing portions 10 is not limited thereto, and for example, only one wearing portion 10 may be provided. As illustrated in FIG. 2, the first sensor 14 and the second sensor 16 may be provided in only one of the wearing portions 10. Alternatively, the first sensor 14 and the second sensor 16 may be provided in both the wearing portions 10. As illustrated in FIG. 2, the awakener 15 and the notifier 17A may be the same output device such as a speaker capable of outputting a sound.

The neck hanging portion 20 is connected to the wearing portions 10. As an example, in a state in which the wearing portion 10 is inserted into the user's ear, the neck hanging portion 20 is positioned around the user's neck. The neck hanging portion 20 includes a controller 11, an input unit 12, a storage 18, and a communicator 19. The neck hanging portion 20 may include a volume button for adjusting a volume, and a chargeable and dischargeable battery. As illustrated in FIGS. 1 and 2, the neck hanging portion 20 may include a notifier 17B.

In the device 1, the wearing portion 10 may have all the configurations of the device 1. In this case, the device 1 may not include the neck hanging portion 20. When the device 1 includes two wearing portions 10, the two wearing portions 10 may be connected to each other by wire via the neck hanging portion 20 or directly. In this case, a time lag between sounds reproduced in each of the two wearing portions 10 can be reduced. When the two wearing portions 10 are connected to each other via the neck hanging portion 20, sizes of the wearing portions 10 can be reduced by providing the controller 11, the input unit 12, and the like in the neck hanging portion 20 as described above.

On the other hand, the device 1 may not include the neck hanging portion 20, and the two wearing portions 10 may be wirelessly connected to each other. In this case, the device 1 does not come into contact with the body of the user at a position other than the position where the wearing portion 10 is worn, and a possibility that the device 1 will come off the body of the user can be reduced.

The controller 11 executes various processing of the device 1. As illustrated in FIG. 1, the controller 11 includes a timer controller 111, a sensor controller 112, a first acquirer 113 (acquirer), a recovery controller 114, an awakening controller 115 (determiner), a second acquirer 116, and a notification controller 117. The respective processing that is executed in the controller 11 will be described in detail below. Upon acquiring, from the input unit 12, a signal indicating that an operation for instructing termination of an operation of the NAP function has been received, the controller 11 terminates the operation and processing of each unit related to the NAP function in the device 1.

The input unit 12 receives an instruction operation from the user. For example, the input unit 12 may be a button capable of receiving an operation for instructing start and end of the NAP function. Upon receiving a user's operation, the input unit 12 outputs a signal corresponding to the operation to the controller 11. For example, upon receiving an operation for instructing start of the operation of the NAP function, the input unit 12 outputs a signal indicating that the operation has been received to the timer controller 111. The input unit 12 outputs, upon receiving an operation for instructing end of the operation of the NAP function, a signal indicating that the operation has been received to the controller 11. The input unit 12 may further include a button capable of receiving an input for instructing switching between ON and OFF states of a power supply of the device 1.

The timer 13 operates under control of the timer controller 111, and measures an elapsed period after the operation of the NAP function is started. In other words, the timer 13 measures an elapsed period after the determination of the awakening timing based on the first index is started in the sleep management system 100.

The first sensor 14 includes an irradiator and a light receiver (not illustrated). The first sensor 14 is a sensor that generates an electric signal having an intensity corresponding to the light received by the light receiver. The irradiator is a light emitting element capable of emitting light having a desired wavelength and intensity under control of the sensor controller 112. The irradiator may be a laser diode capable of emitting laser light. The laser light emitted from the irradiator may have a wavelength of, for example, 700 nm to 900 nm. The light emitted to the user by the irradiator is scattered by blood cells moving together with the blood flow and blood vessels through which blood flows, thereby generating scattered light.

The light receiver is a light receiving element capable of receiving scattered light (optical signal) generated as a result of irradiating the user with the laser light and outputting an electric signal corresponding to the scattered light. The light receiver may be a photodiode that generates an electric signal having an intensity corresponding to the received light. The light receiver outputs the generated electric signal to the first acquirer 113. The light receiver may generate an electric signal each time the scattered light is received. In the case of the device 1 having a shape that can be worn in the ear hole of the user, the irradiator may be disposed at a position where the laser light can be emitted toward an auricle of the user (i.e., toward capillaries of the auricle). The light receiver may be disposed at a position where scattered light can be received from the capillaries receiving the laser light.

The awakener 15 operates under control of the awakening controller 115 and encourages the user to wake up. As an example, the awakener 15 may be an output unit such as a speaker capable of outputting a desired sound under control of the awakening controller 115.

The second sensor 16 is a sensor capable of generating a signal corresponding to a degree of motion of the user. For example, the second sensor 16 may be an acceleration sensor that operates under control of the sensor controller 112 and is capable of generating a signal having an intensity corresponding to a change in posture of the user. The second sensor 16 outputs the generated signal to the second acquirer 116.

The notifier 17 operates under control of the notification controller 117, and performs various notifications to the user. As illustrated in FIG. 2, the device 1 may include, as the notifier 17, the notifier 17A in the wearing portion 10 and the notifier 17B in the neck hanging portion 20. The notifier 17A is an acoustic output unit such as a speaker capable of outputting a sound. The notifier 17B may be an optical output unit such as a light emitting diode (LED) capable of switching a color of light or a pattern of lighting manner in accordance with control of the notification controller 117. The notifier 17A and the awakener 15 may be the same speaker. The device 1 allows the user to recognize the information to be notified by causing the user to listen to the sound output from the notifier 17A or causing the user to view the light pattern of the notifier 17B. The notifier 17 is not limited to the configuration described above. For example, the notifier 17 may be a display device such as a display provided on the neck hanging portion 20 and capable of displaying a desired notification content.

The storage 18 stores various types of information used in the device 1. The storage 18 stores identification information (device ID) for identifying the device 1. The communicator 19 is a communication module for the device 1 to communicate with the terminal device 2. As an example, the communicator 19 may be a module capable of performing short-range wireless communication such as Bluetooth (registered trademark).

The timer controller 111 controls an operation of the timer 13. Specifically, upon acquiring a signal indicating an instruction to start the operation of the NAP function from the input unit 12, the timer controller 111 causes the timer 13 to start measuring the elapsed period. The timer controller 111 causes, upon acquiring information indicating that the operation of the NAP function has ended from the awakening controller 115 or the notification controller 117, the timer 13 to end the measurement. The timer controller 111 outputs, when starting the operation of the timer 13, information indicating that the timer 13 has started measurement to the sensor controller 112.

The sensor controller 112 controls operations of the first sensor 14 and the second sensor 16. Specifically, the sensor controller 112 starts, upon acquiring information indicating that the timer 13 has started the measurement from the timer controller 111, the operation of the first sensor 14. The sensor controller 112 stops, upon acquiring error information indicating that a fatal error has occurred from the recovery controller 114, the operation of the first sensor 14 and starts the operation of the second sensor 16. The sensor controller 112 stops, when the operation of the NAP function ends, the operation of the first sensor 14 or the second sensor 16.

The first acquirer 113 acquires first information for generating the first index. Specifically, the first acquirer 113 acquires a sensor signal from the first sensor 14. The sensor signal indicates, for example, a value reflecting a physiological state related to the blood flow of the user, and corresponds to the first information described above. The first acquirer 113 transmits the sensor signal value together with the device ID to the server device 3 via the communicators 19, 21 and 32.

When a fatal error occurs in the device 1 while the NAP function is operating, the recovery controller 114 acquires information indicating that the error has occurred, and performs control for recovery from the error. When a fatal error occurs, each unit of the device 1 outputs information indicating that the error has occurred to the recovery controller 114.

The fatal error is an error in which the acquisition of the first information or the transmission of the first information cannot be normally performed in the device 1 due to the occurrence of the error. Errors that can occur in the device 1 include an error that is fatal if it occurs once and an error that is allowed if it occurs several times. Specifically, the fatal error is an error in which the acquisition of the first information or the transmission of the first information is not normally performed in the device 1 if the error occurs even once. Alternatively, the case where a fatal error has occurred may mean a case where a slight error causing measurement or transmission of an amount of blood flow not to be normally performed due to occurring multiple times has occurred at a predetermined rate or more within predetermined time exceeding an allowable number of times or a case where the slight error has occurred continuously.

As an example, the fatal error is a calculation timeout error in the first acquirer 113. The calculation timeout error is an error that occurs when the first acquirer 113 performs some calculation on the first information. For example, the calculation timeout error is an error indicating a state in which the first acquirer 113 does not complete calculation processing related to the first information within a predetermined time. The predetermined time is, for example, a period of time from when the first acquirer 113 acquires a signal from the first sensor 14 once to when the first acquirer acquires the signal next time in a normal state. The error as described above occurs when a signal is not output to the first acquirer 113 due to, for example, a malfunction of the first sensor 14 or a contact failure between the first sensor 14 and the controller 11. In this case, the first acquirer 113 cannot calculate the first information at predetermined intervals. When a situation occurs in which the first acquirer 113 cannot calculate the first information at predetermined intervals, the first acquirer 113 recognizes that a calculation timeout error has occurred, and outputs information indicating that the calculation timeout error has occurred to the recovery controller 114.

Alternatively, the fatal error is a transmission busy error. The transmission busy error is an error indicating a state in which the first information cannot be normally output from the first acquirer 113 to the communicator 19. The transmission busy error occurs, for example, when UART communication between the first acquirer 113 and the communicator 19 is in a busy state.

When the calculation timeout error or the transmission busy error occurs as described above, the first acquirer 113 outputs information indicating that the calculation timeout error or the transmission busy error has occurred to the recovery controller 114. Upon acquiring the information indicating that the calculation timeout error or the transmission busy error has occurred, the recovery controller 114 specifies the number of times that the error has occurred within predetermined time. When the calculation timeout error or the transmission busy error has occurred more than the allowable number of times within the predetermined time, the recovery controller 114 determines that a fatal error has occurred in the device 1.

The fatal error may be a device status error. The device status error indicates an error in which the device 1 malfunctions for some reason and cannot operate normally. The reason may be, for example, poor contact, external noise, or static electricity.

The recovery controller 114 monitors a state in the device 1 while the device 1 is operating. When a device status error occurs, each unit in the device 1 outputs information indicating that the device status error has occurred to the recovery controller 114. This allows the recovery controller 114 to recognize that the device status error has occurred in the device 1. When a device status error has occurred even once, the recovery controller 114 determines that a fatal error has occurred in the device 1.

When determining that a fatal error has occurred in the device 1, the recovery controller 114 outputs error information indicating that the fatal error has occurred to the sensor controller 112. The recovery controller 114 may transmit the error information together with the device ID to the server device 3 via the terminal device 2.

When the device 1 or the NAP function is restarted (rebooted) after the fatal error has occurred, the recovery controller 114 determines whether the fatal error has been recovered, and outputs a result of the determination to the notification controller 117. When the fatal error is recovered, in other words, when the sleep management system 100 is normally restored and is in a state in which the first determiner 313 (described below) can normally determine the awakening timing of the user, the recovery controller 114 outputs a result of the determination to each unit of the device 1. This causes the normal operation of the NAP function in the sleep management system 100 to be restarted.

The awakening controller 115 controls the operation of the awakener 15 and can notify the user of appropriate awakening timing. Specifically, the awakening controller 115 receives information indicating the awakening timing of the user from the server device 3 via the terminal device 2. Upon receiving the information, the awakening controller 115 acquires, from the timer 13, information indicating the elapsed period after the operation of the NAP function starts. When the elapsed period acquired from the timer 13 reaches the awakening timing determined by the second determiner 315, the awakening controller 115 operates the awakener 15 to notify the user of the awakening timing. That is, the awakening controller 115 wakes up the user at timing determined based on the elapsed period measured by the timer 13. As described above, the awakening controller 115 can be regarded as a determiner that determines whether the second index has reached the awakening timing to be notified to the user. The awakening controller 115 may cause the awakener 15 to operate and then output information indicating that the awakener 15 is operated, in other words, information indicating that the operation of the NAP function is completed, to each unit of the controller 11. The awakening controller 115 may be provided in the server device 3. In this case, the awakening controller 115 receives information indicating the elapsed period since the NAP function is turned on or the timing at which the NAP function is turned on from the device 1 via the communicator 32. When the elapsed period since the NAP function is turned on reaches the awakening timing, the awakening controller 115 transmits information indicating that the elapsed period as the second index has reached the awakening timing to the device 1 via the communicator 32. Upon receiving the information, the controller 11 of the device 1 controls the awakener 15 to notify the user of the awakening timing.

The second acquirer 116 acquires a signal indicating a degree of motion of the body of the user from the second sensor 16. Upon acquiring the signal, the second acquirer 116 calculates information indicating the degree of motion of the user on the basis of the signal. The second acquirer 116 transmits the calculated information together with the device ID to the server device 3 via the terminal device 2.

The notification controller 117 operates the notifier 17 to perform various notifications to the user. The notification to the user may be a voice guidance or a notification sound by the notifier 17A, or may be a notification by a change in a lighting manner of the notifier 17B. The notification to the user may be performed by another method.

Upon receiving information indicating a determination result to the effect that the user is awake, i.e., the user is not in the sleep state from the server device 3 via the terminal device 2, the notification controller 117 operates the notifier 17 to notify that a fatal error has occurred in the device 1. The notification may include a notification encouraging an operation to restore the sleep management system 100 to a state in which the first determiner 313 is able to make determination. Specifically, the operation is an operation for restarting the device 1 or the NAP function, and may be, for example, an operation such as long-pressing the input unit 12.

If receiving information indicating a determination result to the effect that the user is in the sleep state from the server device 3 via the terminal device 2, the notification controller 117 does not immediately notify the user. In this case, the notification controller 117 operates the notifier 17 after acquiring the information indicating that the awakener 15 is operated from the awakening controller 115, and notifies the user of the occurrence of the fatal error in the device 1. That is, after the awakener 15 notifies the user of the awakening timing, the notification controller 117 notifies that an error has occurred.

When the operation of the device 1 or the NAP function is restarted after the occurrence of the fatal error, the notification controller 117 acquires, from the recovery controller 114, information indicating a determination result as to whether the error is recovered and the device 1 returns to the normal operation state, and notifies the user of the determination result. Specifically, when the device 1 returns from the fatal error by restarting, the notification controller 117 operates the notifier 17 to notify the user that the operation of the device 1 is normally restored.

On the other hand, when the device 1 does not return from the fatal error even if the device is restarted, the notification controller 117 operates the notifier 17 to notify the user that the operation of the device 1 is not normally restored. At this time, the notification controller 117 may perform notification to encourage the user to restart the device 1 again.

### Terminal Device 2

The terminal device 2 relays transmission and reception of various types of information between the device 1 and the server device 3. As an example, the terminal device 2 may be a device such as a smartphone possessed by a user. The terminal device 2 is communicably connected to the device 1 and the server device 3. As illustrated in FIG. 1, the terminal device 2 includes a communicator 21, a controller 22, and a storage 23.

The communicator 21 is a communication module for the terminal device 2 to communicate with the device 1 and the server device 3. For example, the device 1 and the terminal device 2 may be connected to each other by short-range wireless communication such as Bluetooth (registered trademark). For example, the terminal device 2 and the server device 3 may be connected to each other by long-distance wireless communication.

Upon receiving various types of information from the device 1 via the communicator 21, the controller 22 transmits the information to the server device 3. Upon receiving various types of information from the server device 3, the controller 22 transmits the information to the device 1. A variety of control programs for operating the terminal device 2 are stored in the storage 23.

### Server Device 3

The server device 3 determines awakening timing preferable for awakening of the user on the basis of the information on the user acquired by the device 1. When a fatal error occurs in the device 1, the server device 3 determines whether the user is in the sleep state. As an example, the server device 3 may be a cloud server. As illustrated in FIG. 1, the server device 3 includes a controller 31, a communicator 32, and a storage 33, and is communicably connected to the terminal device 2.

The controller 31 controls an operation of each unit of the server device 3 and performs various processing. As illustrated in FIG. 1, the controller 31 includes a generator 311, a first sleep determiner 312, a first determiner 313 (determiner), a second sleep determiner 314 (sleep determiner), and a second determiner 315 (determiner). The communicator 32 is a communication module for the server device 3 to communicate with the terminal device 2.

The storage 33 stores information for performing various processing in the server device 3. As illustrated in FIG. 1, the storage 33 stores a sleep history DB 331 (sleep history database). The sleep history database 331 is a database in which the device ID of the device 1 and history data of a sleep period of the user who uses the device 1 are associated with each other and stored for each device 1. The storage 33 may record an occurrence history of errors that have occurred in the device 1.

The generator 311 generates a first index used in the determination by the first sleep determiner 312 on the basis of the first information. Specifically, the generator 311 receives the first information from the device 1 via the terminal device 2. The generator 311 generates information indicating an amount of blood flow of the user as the first index on the basis of the information, and outputs the information to the first sleep determiner 312. The generator 311 may generate waveform data of a pulse wave of the user, i.e., pattern data or the like indicating a variation pattern of the amount of blood flow of the user for each time, and may use the waveform data as the first index.

Upon acquiring the first index from the generator 311, the first sleep determiner 312 determines (estimates) a sleep stage of the user on the basis of the data. The first sleep determiner 312 outputs information indicating the sleep stage of the user to the first determiner 313.

The sleep stage is an index indicating a depth of sleep of the user. For example, the sleep stage may indicate whether the user is in an awake state or in a sleep state. The sleep stage may be classified in more detail. For example, the sleep stage may include a state in which the user is in light sleep such as rapid eye movement (REM) sleep or a state in which the user is in deep sleep such as non-REM sleep. The non-REM sleep stage may be further classified according to the depth of sleep. For example, the non-REM sleep stage may be classified into stage 1, stage 2, stage 3, and stage 4 in order of decreasing sleep intensity.

Specifically, the first sleep determiner 312 may include a neural network capable of determining the sleep stage from the information indicating the amount of blood flow of the user. In this case, the neural network may be a learned neural network pre-learned using the information indicating the amount of blood flow of the user measured based on the first sensor 14, as input data to be used for learning, and the sleep stage of the user at the time when the information measured based on an electroencephalograph or the like has been generated, as teacher data. The neural network included in the first sleep determiner 312 uses the information indicating the amount of blood flow of the user acquired from the generator 311 as input data and determines a current sleep stage of the user from the input data.

However, the information used by the neural network included in the first sleep determiner 312 is not limited to the information indicating the amount of blood flow of the user. For example, the neural network may be capable of determining the sleep stage of the user on the basis of waveform data indicating a pulse wave of the user. In this case, the neural network may perform learning by using waveform data indicating a pulse wave of the user as input data to be used for learning and a sleep stage of the user at the time when the waveform data has been acquired as teacher data.

Upon acquiring the information indicating the sleep stage of the user from the first sleep determiner 312, the first determiner 313 determines the awakening timing of the user on the basis of whether the sleep stage satisfies the first criterion. In other words, the first determiner 313 can determine the awakening timing of the user on the basis of the first criterion. The first criterion may be a criterion indicating a range of sleep stages suitable for awakening the user. As a specific example, the first criterion may be a criterion indicating a range from stage 2 to stage 3 of non-REM sleep. In this case, the first determiner 313 determines the timing at which the sleep stage of the user becomes a relatively light stage such as stage 2 to stage 3 of non-REM sleep, as the awakening timing of the user. The first determiner 313 transmits information indicating the determined awakening timing of the user to the device 1 via the terminal device 2. The first determiner 313 may further measure a sleep period until the awakening timing of the user. In this case, the sleep period may be time from when the user falls asleep until the awakening timing of the user or time from when the user stands still until the awakening timing of the user. The sleep period measured by the first determiner 313 can be recorded in, for example, the storage 33 described below.

The second sleep determiner 314 can determine whether the user is in the sleep state on the basis of a third index different from the first index. Specifically, the second sleep determiner 314 receives information indicating the degree of motion of the user from the device 1 via the terminal device 2. Upon receiving the information, the second sleep determiner 314 determines whether the user is in the sleep state on the basis of the information. The determination may be made using an algorithm (AW2 equation, Cole equation, or the like) similar to an Actigraph. When determining that the user is awake, the second sleep determiner 314 transmits a result of the determination to the device 1 via the terminal device 2. This causes the notification controller 117 of the device 1 to perform notification to the user on the basis of the result of the determination, allowing the user to recognize the occurrence of the fatal error in the device 1. On the other hand, when determining that the user has already fallen asleep and is in the sleep state, the second sleep determiner 314 outputs a result of the determination to the second determiner 315.

The second determiner 315 can determine the awakening timing of the user on the basis of a second index different from the first index. Specifically, upon acquiring the information indicating the determination result to the effect that the user is in the sleep state from the second sleep determiner 314, the second determiner 315 refers to the storage 33. The second determiner 315 determines (decides) timing at which the elapsed period after the NAP function as the second index is started reaches any one of the elapsed periods included in the sleep history stored in the storage 33, as the awakening timing of the user. For example, the second determiner 315 refers to the sleep history database 331 and specifies the sleep period included in the sleep history of the user who uses the device 1 specified by the device ID received by the server device 3. The second determiner 315 may determine timing at which the second index (elapsed period) reaches a previous sleep period of the user specified on the basis of the sleep history, as the awakening timing of the user. Alternatively, the second determiner 315 may determine timing at which the second index (elapsed period) reaches a statistical value of a past sleep period history of the specified user, as the awakening timing of the user. The past sleep period history of the user may be, for example, an entire history of previous sleep periods of the user. The statistical value of the past sleep periods of the user is time determined on the basis of the past sleep periods of the user stored in the sleep history database 331, and may be, for example, an average value, a median value, or the like of the past sleep periods of the user. When no history of the sleep periods of the user in the sleep history database 331, the second determiner 315 may determine timing within a range of 15 minutes or more and 30 minutes or less from the start of the operation of the timer 13, as the awakening timing of the user. That is, the elapsed period used as a criterion for determination in the second determiner 315 may be information related to the sleep period. Specifically, the elapsed period may be any one of (1) time within a range of 15 minutes or more and 30 minutes or less, (2) a previous sleep period of the user, or (3) a statistical value of past sleep periods of the user. The second determiner 315 may determine, as the awakening timing of the user, timing obtained by adding a period of time considered to have elapsed until the user falls asleep, for example, five minutes to the above-described time. The second determiner 315 transmits information indicating the determined awakening timing of the user to the device 1 via the terminal device 2.

### Example of Processing Flow During Normal Operation

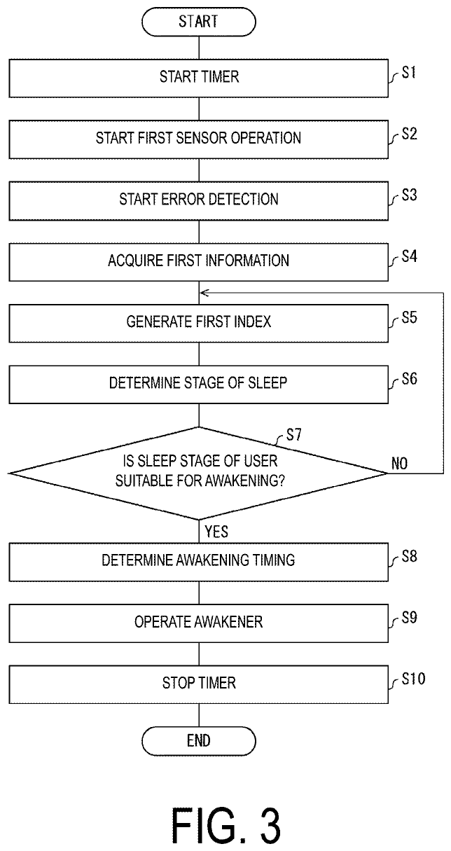

FIG. 3 is a flowchart illustrating an example of a flow of an operation (sleep management method) of a NAP function performed in the sleep management system 100. The example illustrated in FIG. 3 illustrates processing and an operation (normal operation) performed when no fatal error occurs in the device 1. The example of a flow of the normal operation of the NAP function performed in the sleep management system 100 will be described below with reference to FIG. 3.

First, the controller 11 receives an input instruction to start an operation of the NAP function from the user via the input unit 12. Upon receiving the instruction, the timer controller 111 controls the timer 13 to start measuring an elapsed period after the NAP function starts (S1). The timer controller 111 outputs, to the sensor controller 112, information indicating that the timer 13 has started measuring the time.

Upon acquiring the information indicating that the timer 13 has started measuring the time, the sensor controller 112 starts the operation of the first sensor 14 (S2). Specifically, the sensor controller 112 causes the irradiator of the first sensor 14 to start irradiation with laser light. This causes scattered light generated by reflection of the light emitted to the user is incident on the light receiver. The light receiver generates a signal having an intensity corresponding to the scattered light, and outputs the signal to the first acquirer 113. The sensor controller 112 outputs information indicating that the operation of the first sensor 14 has been started to the recovery controller 114.

Upon acquiring the information indicating that the operation of the first sensor 14 has started, the recovery controller 114 starts monitoring whether a fatal error has occurred in the device 1, in other words, starts detecting occurrence of a fatal error (S3).

When the operation of the first sensor 14 starts in step S2, a signal having an intensity corresponding to the physiological state of the user is input to the first acquirer 113. The first acquirer 113 acquires the signal as first information (S4). The first acquirer 113 transmits the first information together with the device ID to the server device 3 via the terminal device 2.

The generator 311 of the server device 3 receives information indicating the first information from the device 1 via the terminal device 2. The generator 311 generates information indicating the amount of blood flow of the user as the first index on the basis of the information (S5), and outputs the information to the first sleep determiner 312.

Upon acquiring the first index, the first sleep determiner 312 determines the sleep stage of the user on the basis of the first index (S6). The first sleep determiner 312 outputs information indicating the determined sleep stage of the user to the first determiner 313.

Upon acquiring information indicating a result of the determination by the first sleep determiner 312, i.e., the sleep stage of the user, the first determiner 313 determines whether the acquired sleep stage of the user satisfies the first criterion, i.e., whether the acquired sleep stage of the user is a sleep stage suitable for awakening of the user (S7). The first determiner 313 repeats the determination until it determines that the sleep stage of the user is a sleep stage suitable for awakening of the user. That is, when the sleep stage of the user is not a sleep stage suitable for awakening of the user (NO in S7), the processing in the sleep management system 100 returns to S5, and the processing from S5 to S7 is repeated. However, the processing of S5 performed after S7 uses the first information newly received from the device 1.

When the sleep stage of the user is a sleep stage suitable for awakening of the user (YES in S7), the first determiner 313 determines the timing of making the determination as the determination result of the suitable awakening timing of the user. In other words, the first determiner 313 determines the awakening timing of the user on the basis of the first index (S8: first determination step). The first determiner 313 specifies the device 1 of a transmission destination of the information on the basis of the device ID, and transmits information indicating the determined awakening timing of the user to the device 1 via the terminal device 2.

Upon receiving the information indicating the awakening timing of the user determined by the first determiner 313, the awakening controller 115 of the device 1 operates the awakener 15 to notify the user of the awakening timing (S9). That is, the awakener 15 notifies the user of the awakening timing on the basis of the determination result of the sleep stage of the user by the first determiner 313. Specifically, the awakening controller 115 causes a speaker as the awakener 15 to output a sound such as an alarm. This encourages the user to wake up at appropriate timing. After operating the awakener 15, the awakening controller 115 outputs information indicating that the awakening timing has been notified to the user to each unit of the controller 11, for example, the timer controller 111 and the sensor controller 112.

Upon acquiring the information, the sensor controller 112 ends the operation of the first sensor 14. Upon acquiring the information, the timer controller 111 causes the timer 13 to stop measuring the elapsed period (S10). Thereby, the operation of the NAP function in the sleep management system 100 is completed.

### Example of Processing Flow When Error Occurs

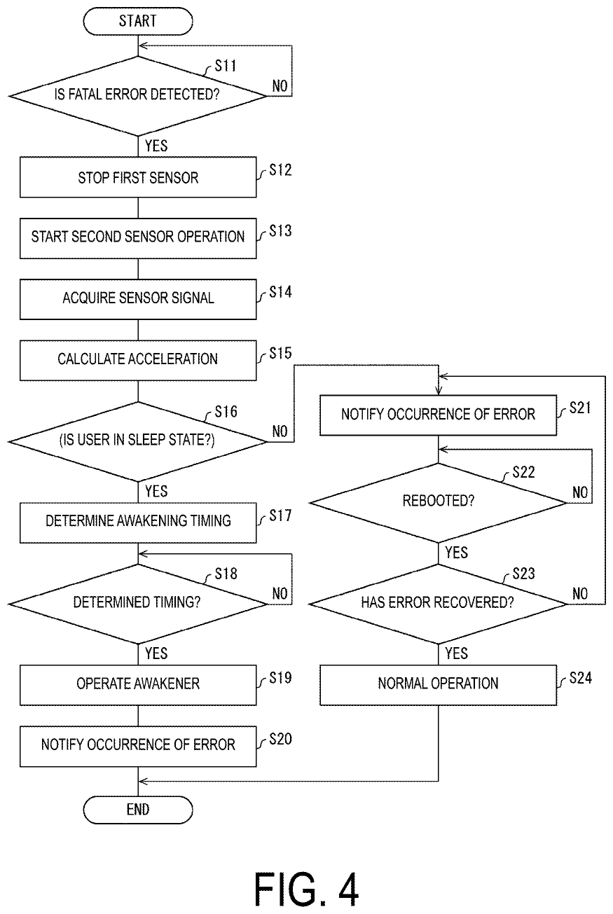

FIG. 4 is a flowchart illustrating another example of the flow of the operation of the NAP function performed in the sleep management system 100. The example illustrated in FIG. 4 illustrates processing and an operation performed when a fatal error occurs in the device 1. The example of a flow of operation of the occurrence of error of the NAP function performed in the sleep management system 100 will be described below with reference to FIG. 4.

First, during the normal operation of the NAP function described above, the recovery controller 114 repeatedly determines whether a fatal error has occurred in the device 1 (S11). In other words, the recovery controller 114 monitors occurrence of a fatal error in the device 1. When a fatal error has occurred in the device 1 (YES in S11), in other words, when an error has occurred in the process of determination made by the first determiner 313, the recovery controller 114 starts control for recovery from the error. For example, when information indicating that an error that a signal from the first sensor 14 is not normally acquired has occurred is acquired from the first acquirer 113, the recovery controller 114 starts control for recovery from the error.

Specifically, the recovery controller 114 outputs information indicating that a fatal error has occurred to the sensor controller 112. Upon acquiring the information, the sensor controller 112 stops the operation of the first sensor 14 (S12) and starts the operation of the second sensor 16 (S13). This causes the second acquirer 116 to start acquiring the signal output from the second sensor 16 (S14). Upon acquiring the signal, the second acquirer 116 calculates an index related to the motion of the user, for example, an acceleration indicating the degree of the motion of the user on the basis of the signal (S15), and acquires the index as a third index. The second acquirer 116 transmits information indicating the calculated degree of the motion of the user together with the device ID to the server device 3 via the terminal device 2.

Upon receiving the information indicating the degree of the motion of the user, the second sleep determiner 314 of the server device 3 determines whether the user is in the sleep state on the basis of the information (S16).

When determining, in S16, that the user is in the sleep state (YES in S16), the second sleep determiner 314 outputs information indicating a result of the determination to the second determiner 315. Upon acquiring the information, the second determiner 315 refers to the storage 33 and determines the awakening timing of the user based on the second index (S17: second determination step). Specifically, the second determiner 315 refers to the sleep history database 331 on the basis of the device ID, and specifies the history of the sleep period of the user who uses the device 1 specified by the device ID. The second determiner 315 may determine the timing at which the second index (elapsed period) reaches the previous sleep period of the user as the determination result indicating the awakening timing of the user. Alternatively, the second determiner 315 may determine the timing at which the second index (elapsed period) reaches the statistical value of the history of the previous sleep period of the user as the determination result indicating the awakening timing of the user. When there is no history of the sleep period of the user in the sleep history database 331, the second determiner 315 may determine the timing at which the second index reaches time within the range of 15 minutes or more and 30 minutes or less as the determination result indicating the awakening timing of the user.

The second determiner 315 specifies the device 1 of a transmission destination on the basis of the device ID, and transmits information indicating the determined awakening timing of the user to the device 1 via the terminal device 2. The awakening controller 115 of the device 1 acquires, upon receiving the information, the elapsed period after the operation of the NAP function starts from the timer 13 and waits until the awakening timing indicated by the information arrives. In other words, the awakening controller 115 determines whether the elapsed period indicated by the timer 13 has reached the awakening timing (S18). When the elapsed period indicated by the timer 13 has reached the awakening timing (YES in S18), the awakening controller 115 operates the awakener 15 (S19) to notify the user of the awakening timing. This encourages the user to wake up at appropriate timing.

The awakening controller 115 outputs information indicating that the awakener 15 has been operated to each unit of the controller 11. Upon acquiring the information, the notification controller 117 operates the notifier 17 to notify the user that an error has occurred in the device 1 (S20).

On the other hand, when determining that the user is in an awake state (NO in S16), the second sleep determiner 314 transmits information indicating a result of the determination to the device 1 via the terminal device 2. Upon receiving the information, the notification controller 117 of the device 1 controls the notifier 17 to notify the user of information to the effect that a fatal error has occurred in the device 1 (S21). The notification includes a notification to encourage the user to operate to restore the sleep management system 100 to a state in which the first determiner 313 is able to make determination based on the first index, i.e., to operate to restore (reboot) the device 1.

After S21, the recovery controller 114 determines whether the device 1 is restarted (S22). If NO in S22, the recovery controller 114 repeats the processing of S22. When the device 1 is restarted after the above-described notification is performed (YES in S22), the recovery controller 114 determines whether the fatal error detected in S11 has been recovered (S23). When the device 1 is restarted to recover the fatal error (YES in S23), the recovery controller 114 determines that the sleep management system 100 is normally restored and is in a state in which the first determiner 313 can make the determination normally, and outputs a result of the determination to each unit of the controller 11. This causes the sleep management system 100 to restart the normal operation of the NAP function (S24). Specifically, the operation after S1 illustrated in FIG. 3 is started. Therefore, the first determiner 313 can start determining the awakening timing of the user based on the first index.

If NO in S23, i.e., if the error is not recovered even when the device 1 is restarted, the sleep management system 100 repeats the operation after S21.

### Effects of Sleep Management System 100

As described above, the sleep management system 100 includes the first determiner 313 capable of determining the awakening timing of the user, and the awakener 15 capable of notifying the user of the awakening timing on the basis of the determination result of the first determiner 313. The sleep management system 100 includes the second determiner 315 capable of determining the awakening timing of the user on the basis of the second index when a trouble occurs (a fatal error occurs) in the determination of the awakening timing made by the first determiner 313 on the basis of the first index.

According to the above configuration, even when an error occurs in the device 1 and a trouble occurs in the determination based on the first index, the user can be notified of the awakening timing at timing estimated to be preferable.

The second index used in the second determiner 315 may be the elapsed period after the determination of the awakening timing based on the first index is started, i.e., the elapsed period after the timer 13 starts measurement after the NAP function is turned on. According to the above configuration, the sleep management system 100 can determine the awakening timing of the user on the basis of the elapsed period even when a trouble occurs in the determination by the first determiner 313.

The sleep management system 100 may include the second sleep determiner 314. When the first determiner 313 cannot make the determination based on the first index, the second sleep determiner 314 may determine whether the user is in the sleep state on the basis of the third index different from the first index.

According to the above configuration, the sleep management system 100 can determine whether the user is in the sleep state on the basis of the third index and determine the awakening timing of the user on the basis of the third index when a trouble occurs in the determination of the awakening timing based on the first index.

The third index may be an index related to a motion of the user. This allows, when the sleep determination cannot be made based on the first index, the sleep management system 100 to make sleep determination based on the index (for example, acceleration) related to the motion of the user.

The sleep management system 100 may include the notifier 17, and the notifier 17 may perform notification that a trouble occurs when the second sleep determiner 314 determines that the user is not in the sleep state. According to the above configuration, the notification by the notifier 17 is not performed while the user is sleeping. Therefore, the user's sleep can be suppressed from being disturbed due to the notification by the notifier 17.

The notifier 17 may perform notification that a trouble occurs after the awakener 15 notifies the user of the awakening timing. According to the above configuration, the notification by the notifier 17 is not performed while the user is sleeping, and the notification can be performed after the awakening timing is notified to the user, i.e., at timing at which the user is considered to be awake. Therefore, the occurrence of the error can be notified without disturbing the user's sleep.

The notification that an error has occurred may include a notification to encourage the user to operate to restore the sleep management system 100 to a state in which the first determiner 313 is able to make determination. The above configuration can encourage, when the user is not in the sleep state or after the user is encouraged to be awakened, the user to operate for restoration and restore the system without disturbing the user's sleep.

When the sleep management system 100 is normally restored so that the first determiner 313 can make determination, the notifier 17 may perform notification of that effect, and the first determiner 313 may start determining whether the user is in the sleep state.

According to the above configuration, when the sleep management system 100 returns to a state in which the first determiner 313 can make determination by an operation of the user, the sleep determination can be made after causing the user to recognize the return.

The sleep management system 100 may include the timer 13 that measures an elapsed period after the determination based on the first index is started, and the awakener 15 may awaken the user at timing determined based on the elapsed period measured by the timer 13.

According to the above configuration, even when the determination based on the first index is not made, the user can be notified of the awakening timing at timing calculated with the timing at which the determination is started, i.e., the timing at which the sleep management is started as the starting point.

The sleep management system 100 may include the first acquirer 113 that acquires the first information (for example: amount of blood flow) for generating the first index, and the generator 311 that generates the first index on the basis of the first information.

According to the above configuration, the sleep management system 100 can determine the sleep state of the user on the basis of the first index generated using the first information acquired by the first acquirer 113.

The first acquirer 113 may acquire the information on the blood flow in the body of the user, and the first sleep determiner 312 may use the information on blood flow or information (pulse wave) generated from the information, as the first index. According to the above configuration, the first determiner 313 can determine the awakening timing on the basis of the information on the blood flow of the user or the information generated from the information.

The sleep management system 100 may include the device 1 that includes the first acquirer 113 and is worn on the body of the user, and the server device 3 that includes the first sleep determiner 312 and is separate from the device. According to the above configuration, by implementing the device 1 worn on the body of the user and the server device 3 including the first sleep determiner 312 as separate devices, the size of the device can be reduced.

The second determiner 315 may determine the awakening timing on the basis of whether the second index reaches any of (1) time within a range of 15 minutes or more and 30 minutes or less, (2) a previous sleep period of the user, and (3) a statistical value of past sleep periods of the user.

In general, 15 minutes to 30 minutes is a period of time considered to be suitable for a nap. The above configuration can encourage the user to wake up at timing suitable for the user on the basis of the history of sleep of the user. Even when there is no history of sleep of the user, the user can be notified of the awakening timing after the elapse of at least a period of time generally considered to be preferable for a nap.

In the embodiment described above, the example has been described in which the criterion used for the above-described determination by the second determiner 315 is based on the data of the past sleep period of the user who uses the device 1. However, in the determination made by the second determiner 315, the data of the past sleep period of another user who uses another device 1 may also be taken into consideration.

### Variations

A lower limit value, for example, 2 minutes may be set for the awakening timing that is determined by the first determiner 313. This allows, even when the user enters a sleep stage that can be determined to be awakening timing as soon as the user falls asleep, the user not to be notified of the awakening timing at that time. This can reduce a possibility of an excessively short napping period of the user.

In the sleep management system 100, the storage 23 of the terminal device 2 may store content information such as music that encourages the user to fall asleep. In this case, after the operation of the NAP function is started in the sleep management system 100, the device 1 may receive the content information from the terminal device 2, and the notification controller 117 may control the notifier 17A to output the content information.

The server device 3 may manage the number of errors that have occurred in the device 1. Specifically, when the number of times of error occurrence is equal to or greater than a preset allowable number of times in a certain device 1, the server device 3 transmits information to encourage to repair or replace the device 1 to the device 1 via the terminal device 2. When the device 1 receives the information, the notification controller 117 may control the notifier 17 to notify the user of the information.

In the sleep management system 100, an upper limit may be set for the number of restarts of the device 1 in one operation of the NAP function. In this case, when a fatal error occurs in the device 1, the recovery controller 114 may specify the number of times that the device 1 has been restarted. When the number of times exceeds a preset upper limit number of times, the recovery controller 114 outputs information to the effect that recovery from an error has not been possible to the notification controller 117. Upon acquiring the information, the notification controller 117 controls the notifier 17 to notify the user of the information.

The information used for the determination by the second determiner 315 is not limited to that described above. For example, in the sleep history database 331, identification information indicating a user and past wake-up time of the user or a statistical value thereof may be recorded in association with each other. In this case, the second determiner 315 may determine timing at which the time specified based on the second index reaches the past wake-up time of the user or the statistical value thereof, as the awakening timing of the user.

The second sleep determiner 314 and the second determiner 315 may operate even when the first determiner 313 can normally make determination. That is, even when no error occurs in the device 1, the device 1 may transmit the information indicating the third index acquired from the second sensor 16 to the server device 3. In this case, the second sleep determiner 314 may determine whether the user is sleeping on the basis of the third index, and the second determiner 315 may determine the awakening timing of the user on the basis of a result of the determination. Further, the storage 33 may store information in which the awakening timing (first awakening timing) of the user determined by the first determiner 313 and the awakening timing (second awakening timing) of the user determined by the second determiner 315 are associated with each other. When a fatal error occurs in the device 1, the second determiner 315 may refer to the storage 33 and determine how many minutes after the second sleep determiner 314 determines that the user falls asleep as the awakening timing on the basis of the above correspondence relationship. That is, the second determiner 315 may correct the second awakening timing determined at the current point in time on the basis of the relationship between the first awakening timing and the second awakening timing already recorded.

As described above, the determination by the first determiner 313 and the determination by the second determiner 315 may be made at the same time. In this case, in the sleep management system 100, the user may be notified of the awakening timing on the basis of at least one of the determination result by the first determiner 313 and the determination result by the second determiner 315.

When an error occurs in the device 1 and the second sleep determiner 314 determines that the user is not in the sleep state, the notification controller 117 may notify the user of the occurrence of the error after notifying the user of the awakening timing. If an error is notified before the user falls asleep and the user performs an operation of returning the device 1, the user's sleep period may not be sufficiently secured. As described above, by notifying the occurrence of the error after encouraging the user to wake up, the concern of shortened sleep period can be reduced.

### Second Embodiment

Another embodiment of the present disclosure will be described below. For the sake of convenience of description, members having the same functions as those of the members described in the above-described embodiment are denoted by the same reference signs, and description thereof is not repeated.

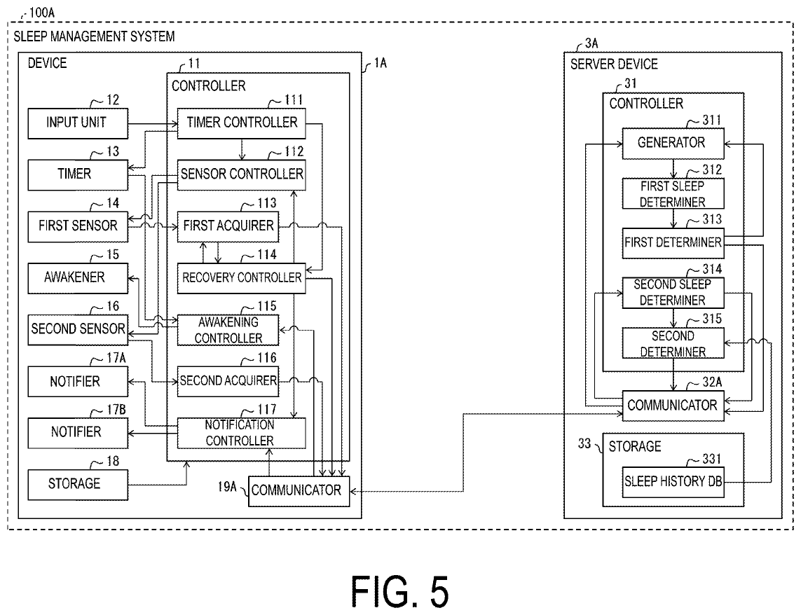

FIG. 5 is a block diagram illustrating a configuration of a sleep management system 100A according to another embodiment. As illustrated in FIG. 5, the sleep management system 100A may be configured to include a device 1A and a server device 3A and not to include the terminal device 2. In this case, a communicator 19A of the device 1A is communicably connected to a communicator 32A of the server device 3A. In other words, in the sleep management system 100A, the device 1A and the server device 3A can directly communicate with each other.

### Third Embodiment

Another embodiment of the present disclosure will be described below. For the sake of convenience of description, members having the same functions as those of the members described in the above-described embodiment are denoted by the same reference signs, and description thereof is not repeated.

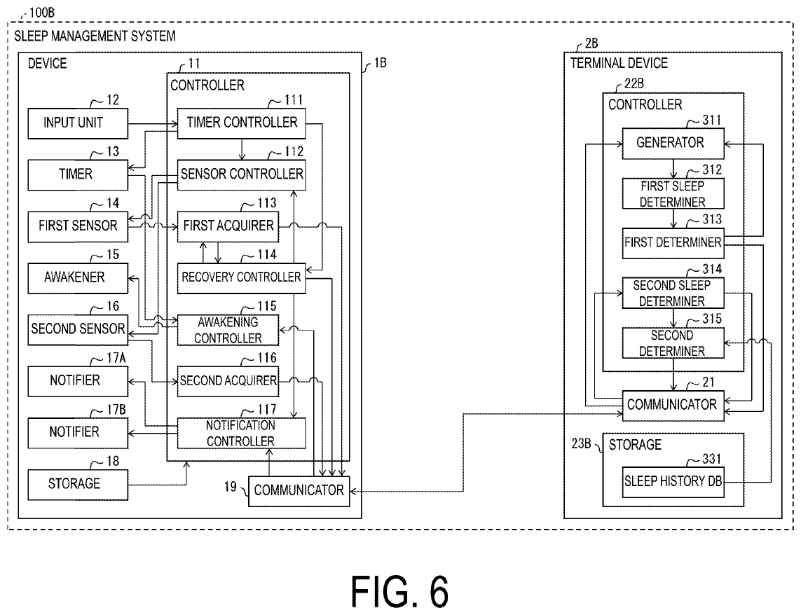

FIG. 6 is a block diagram illustrating a configuration of a sleep management system 100B according to another embodiment. As illustrated in FIG. 6, the sleep management system 100B may be configured to include a device 1B and a terminal device 2B and not to include the server device 3. As illustrated in FIG. 6, the terminal device 2B may include each unit included in the server device 3 described in the first embodiment. Specifically, as illustrated in FIG. 6, the terminal device 2B includes a controller 22B and a storage 23B. The controller 22B includes the generator 311, the first sleep determiner 312, the first determiner 313, the second sleep determiner 314, and the second determiner 315. The storage 23B stores the sleep history database 331.

In the sleep management system 100B, the processing performed by the server device 3 described in the first embodiment, such as determination of the sleep stage of the user based on each piece of information acquired in the device 1 and determination of the awakening timing, may be performed by the terminal device 2B.

### Fourth Embodiment

Another embodiment of the present disclosure will be described below. For the sake of convenience of description, members having the same functions as those of the members described in the above-described embodiment are denoted by the same reference signs, and description thereof is not repeated.

In each of the embodiments described above, the sleep management systems 100, 100A and 100B are each configured by two or more devices. However, the configuration of the sleep management systems 100, 100A and 100B is not limited thereto, and may be configured by only one device. For example, the sleep management systems 100, 100A, and 100B may be each configured to include only the device 1. In this case, each configuration of the server device 3 is provided in the device 1, and each processing related to the NAP function can be completed only by the device 1.

### Example of Software Implementation

Functions of the sleep management system 100, 100A, 100B (hereinafter, referred to as “system”) can be implemented by a control program for causing a computer to function as the system and for causing the computer to function as each control block (in particular, each unit included in the controller 11, 22, 22B, 31) of the system.

In this case, the system includes a computer including at least one control device (e.g., processor) and at least one storage device (e.g., memory) as hardware for executing the control program. By executing the control program by the control device and the storage device, the respective functions described in the respective embodiments are implemented.

The control program may be recorded on one or more computer-readable non-transitory recording media. The recording media may or may not be included in the device. In the latter case, the control program may be supplied to the device via any wired or wireless transmission medium.

Some or all of the functions of the control blocks can be implemented by logic circuits. For example, an integrated circuit in which logic circuits functioning as the control blocks are formed is also included in the scope of the present disclosure. In addition to this, for example, a quantum computer can implement the functions of the control blocks.

The present disclosure is not limited to each of the embodiments described above, and various modifications can be made within the scope indicated by the claims, and an embodiment obtained by appropriately combining technical means disclosed in different embodiments is also included in a technical scope of the present disclosure.

### REFERENCE SIGNS

1, 1A, 1B: Device (first device)
3, 3A: Server device (second device)
13: Timer
15: Awakener
100, 100A, 100B: Sleep management system
113: First acquirer (acquirer)
115: Awakening controller (determiner)
311: Generator
313: First determiner (determiner)
314: Second sleep determiner (sleep determiner)
315: Second determiner (determiner)

1. A sleep management system, comprising:
a determiner configured to determine an awakening timing of a user based on a first index; and
an awakener configured to notify the user of the awakening timing in accordance with a determination result of the determiner,
wherein the determiner determines the awakening timing in accordance with a second index when a problem occurs in a determination of the awakening timing based on the first index.

2. The sleep management system according to claim 1, wherein the second index is an elapsed period.

3. The sleep management system according to claim 1, further comprising a sleep determiner configured to determine whether the user is in a sleep state in accordance with a third index different from the first index when the determiner is not able to make the determination.

4. The sleep management system according to claim 3, wherein the third index is related to a motion of the user.

5. The sleep management system according to claim 3, further comprising a notifier configured to perform a notification that the problem has occurred when the sleep determiner determines that the user is not in the sleep state.

6. The sleep management system according to claim 5, wherein the notifier notifies the user of an occurrence of the problem after the awakener notifies the user of the awakening timing.

7. The sleep management system according to claim 5, wherein the notification that the problem has occurred comprises a notification to the user to restore the sleep management system to a state in which the determiner is able to make the determination.

8. The sleep management system according to claim 7,
wherein when the sleep management system is restored to the state in which the determiner is able to make the determination, the notifier performs a notification of a restored state, and
the determiner starts determining the awakening timing of the user based on the first index.

9. The sleep management system according to claim 1, further comprising
a timer configured to measure an elapsed period after the determination based on the first index,
wherein the awakener awakens the user at timing determined in accordance with the elapsed period measured by the timer.

10. The sleep management system according to claim 1, further comprising:
an acquirer configured to acquire first information for generating the first index; and
a generator configured to generate the first index in accordance with the first information.

11. The sleep management system according to claim 10,
wherein the acquirer acquires information on a blood flow in a body of the user, and
the determiner uses, as the first index, the information on the blood flow or other information generated from the information on the blood flow.

12. The sleep management system according to claim 10, comprising:
a first device comprising the acquirer and configured to be worn on a body of the user; and
a second device comprising the determiner and separate from the first device.

13. The sleep management system according to claim 1, wherein the awakening timing is determined in accordance with whether the second index reaches any one of:
(1) a time within a range of 15 minutes or more and 30 minutes or less;
(2) a previous sleep period of the user; and/or
(3) a statistical value of past sleep periods of the user.

14. A sleep management method in a sleep management system comprising:
determining, by a determiner, an awakening timing of a user in accordance with a first index; and
determining the awakening timing of the user in accordance with a second index when a problem occurs in the determining of the awakening timing based on the first index.

15. A non-transitory computer-readable recording medium that stores a control program for a device, the control program causing the device to execute an individual step of the sleep management method according to claim 14.

16. (canceled)
